(19) United States
(12) Patent Application Publication
(10) Pub. No.: US 2004/0235879 A1
(43) Pub. Date: Nov. 25, 2004

(54) Method for treatment of chemotherapy-induced diarrhea

(76) Inventor: Joseph A. Ware, Kalamazoo, MI (US)

Correspondence Address: Patrick G. Gattari, McDonnell Boehnen Hulbert & Berghoff LLP, 32nd Floor, 300 S. Wacker Drive, Chicago, IL, 60606, US

(21) Appl. No.: 10/850,070

(22) Filed: May 20, 2004

Related U.S. Application Data
(60) Provisional application No. 60/472,348, filed on May 21, 2003.

(51) Int. Cl. A61K 31/4745; A61K 33/24

(52) U.S. Cl. 514/283; 514/50; 424/649

(57) ABSTRACT

The present invention provides a method for treating diarrhea caused by the interaction of a chemotherapeutic agent with a CFTR protein, which comprises administering sequentially, separately or simultaneously with said chemotherapeutic agent a therapeutically effective amount of a CFTR protein inhibitor to a patient in need of the treatment of such a diarrhea and a method for optimizing time and dosages of a diarrheagenic chemotherapeutic agent in a patient in need thereof, which comprises evaluating the sensitivity of said patients towards said agent through the detection of chloride levels in a biological sample of said patient and selecting a time and dosages of said agent based on the above chloride levels.

### FIELD OF THE INVENTION

The present invention relates to the treatment of diarrhea induced by pharmacologically active agents, in particular chemotherapeutic agents.

### DESCRIPTION OF THE INVENTION

Chemotherapy-induced diarrhea (CID) is a prevalent and severe toxicity associated with a variety of sole agents and combinations chemotherapy regimens used for the treatment of cancer. Not only does the occurrence of diarrhea reduce the quality of life of cancer patients, it may also strongly impact the clinical decision about chemotherapy regimens so that patients may undergo changes in treatments. These changes include dose reductions, delays in administration of subsequent drug cycles, and a total discontinuation of therapy. Acute or persistent diarrhea can often increase the cost of care when conventional treatment is unsuccessful and hospitalization is required.

Some chemotherapeutic drugs are more likely than others to cause diarrhea. Cisplatin and 5-fluorouracil, when administered alone, commonly cause diarrhea, as does irinotecan. About 8% of patients receiving 5-fluorouracil alone have severe or life-threatening diarrhea; the frequency increases to up to 25% when 5-fluorouracil is combined with leucovorin. Irinotecan causes severe diarrhea that may be life threatening. Irinotecan-induced diarrhea typically occurs 2 to 14 days after therapy. Several Phase II clinical studies report incidences of delayed CID as high as 80% with irinotecan and more than 18% of patients treated with CPT-11 required hospitalization because of diarrhea occurring alone or in combination with other gastrointestinal disturbances.

Although marked progress has been accomplished these last years, standardized and universally accepted methods for management of CID have not been developed. As a matter of fact, management of CID still represents a formidable challenge to the healthcare team and a substantial degree of further improvement is needed to ameliorate the effectiveness of specific treatments.

According to Kornblau et al. (J. of Pain and Symptom Management, Vol. 19, No. 2, February 2000, pp 118-129), many factors contribute to diarrhea in the cancer patient, including damage to and maturational arrest of intestinal epithelium, inflammation, infection, and antibiotics. Although the pathophysiologic mechanisms for CID are not completely known, histopathologic evidence indicates that diarrhea is probably a multifactorial process that results in absorptive and secretory imbalance in the small bowel. Pathophysiologic mechanisms for cancer treatment-related diarrhea differ based on the causative factor.

According to Ippoliti (Am. J. of Health-System Pharm., Vol. 55(15), Aug. 1, 1998, pp 1573-1580), diarrhea can be classified as osmotic, secretory, exudative, postresection, and motor. Osmotic diarrhea results from the ingestion of poorly absorbed substances that retard fluid absorption.

Both postresection and exudative diarrheas result from loss of functional intestinal mucosa: in exudative diarrhea, the mucosa is damaged by disease; in postresection diarrhea, some amount of functional mucosa is surgically removed.

Abnormally rapid transit time (causing reduced exposure of luminal contents to the intestinal wall) can result in motor diarrhea.

Inhibition of mucosal absorption or stimulation of secretion of fluid and electrolytes results in secretory diarrhea. Within the intestine, immature crypt cells secrete fluid into the lumen, and villi cells absorb fluid from the lumen. Secretory diarrhea can occur when either of these processes is disrupted and there is a net flow of fluid into the lumen. Movement of fluid across the crypt and villi cells is controlled chiefly by membrane-associated proteins involved in epithelial ion transport and smooth muscle contraction. These proteins, in turn, are regulated by secondary messengers, including cyclic nucleotides, elements of the phosphoinositide-diacylglycerol pathway, and free intracellular calcium.

Several studies suggest that cystic fibrosis transmembrane conductance regulator (CFTR), a member of the ATP-binding Cassette (ABC), subfamily C member 7 (ABCC7) is the final common pathway for intestinal chloride (Cl⁻) and thus fluid secretion into the lumen of the small and large intestine. Activation of CFTR (ABCC7) by pathogenic microorganisms is a major factor in enterotoxin-induced diarrhea (EID) produced by many gut pathogens. In many examples, second messengers generated in response to an enterotoxin exposure have been shown to activate CFTR and thus Cl⁻ secretion. These second messengers include cAMP and cGMP protein kinase C, inflammatory mediators (such as tumor necrosis factor-α and interleukin 1 and 8), as well as prostaglandins that are known metabolites of arachidonic acid (such as PGE₂). Despite the complex nature of events leading to ultimate effect of EID, the role of CFTR has been established using in-vitro studies and in mice where CFTR has been selectively deleted from the mouse.

The interaction of chemotherapeutic agents with ABC transporters has been well established with the earliest research describing the role of ABCB1 also known as P-glycoprotein (P-gp) or multidrug resistance gene (MDR1) in the genesis of tumor drug resistance. Further research on acquired drug resistance in tumor cells identified other ABC proteins such as MRP1 (ABCC1), MRP5 (ABCC5), and BCRP/MXR/ABCP (ABCG2). Through the utilization of acquired drug resistance and through the expression of individual clones for these ABC transporters, prototypical substrates have been identified. However, it is important to recognize that there is much overlap in the ability of ABC transporters to interact with drugs or model investigative compounds. For instance, irinotecan has been shown to be a substrate of P-gp (ABCB1), MRP1 (ABCC1), MRP2 (ABCC2), and BCRP/MXR/ABCP (ABCG2). It is likely that interactions may occur between drugs and with other ABC proteins that are responsible for critical physiological functions.

Irinotecan [1,4′-Bipiperidine]-1′-carboxylic acid (4S)-4,11-diethyl-3,4,12,14-tetrahydro-4-hydroxy-3,14-dioxo-1H-pyrano[3′,4′:6,7]indolizino[1,2-b]quinolin-9-yl ester is a camptothecin analog and topoisomerase-I inhibitor derived from a compound, which occurs naturally in the Chinese tree, Camptotheca acuminata. Irinotecan can be prepared following the procedure disclosed in U.S. Pat. No. 4,604,463, European patent No. 835,257 or S. Sawada et al., Chem. Pharm. Bull. 39, 1446 (1991). Irinotecan hydrochloride, clinically investigated as CPT-11, is a commercially available compound (CAMPTOSAR™, Pharmacia Corp.).

As used herein the term “irinotecan” encompasses all pharmaceutically acceptable salts of irinotecan, particularly the hydrochloride salt.

In a preferred aspect of the present invention, irinotecan is irinotecan hydrochloride, namely CPT-11.

Irinotecan is a prodrug extensively metabolized in the liver to various metabolites. It is cleaved enzymatically by carboxylesterases to form 7-ethyl-10-hydroxycamptothecin (SN-38), which has cytotoxic activity that is 100 to 1,000 times greater than that of the parent drug. SN-38 is further conjugated to an inactive glucuronide (SN-38G) by uridine diphosphate glucuronosyltransferases. Other CPT-11 metabolites identified are the major plasma metabolite 7-ethyl-10-[4-N-(5-aminopentanoic acid)-1-piperidino]-carbonyloxycamptothecin (APC) and 7-ethyl-10-[4-amino-1-piperidino]-carbonyloxycamptothecin (NPC), resulting from a ring-opening oxidation of the terminal piperidine ring of CPT-11 mediated by cytochrome P450 3A4 enzymes. The major dose-limiting nonhematologic toxicity of irinotecan is diarrhea. Although the pharmacokinetic-pharmacodynamic relationship between exposure and toxicity has been widely studied, the results have been discrepant because of its complex and incompletely understood relationship and because of different study designs. Although most investigations have reported that area under the concentration-time curve (AUC) values for both irinotecan and SN-38 significantly correlated with the severity of diarrhea, others have detected that correlation only for irinotecan AUC. Similarly, although several studies have suggested that there is a correlation between the biliary index and diarrhea, other studies could not confirm that correlation. Finally, some trials failed to find any relationship between diarrhea and any of the studied pharmacokinetic parameters (Clinical Pharmacology and Therapeutics, Vol. 72(3), September 2002, pp 265-275).

It has now been found that camptothecin derivatives, especially irinotecan and its active metabolite SN-38 would produce disturbances in colonic electrolyte transport by interacting with CFTR (ABCC7) in the colonic crypts, so contributing to diarrhea associated with the administration of said drug in a manner analogue to EID.

As an example, to determine the interaction of CPT-11, SN-38, and topotecan with CFTR (ABCC7), the effect of said substances on Cl⁻ conductance in CFTR (ABCC7)-transfected Xenopus laevis oocytes is evaluated via single voltage clamp conditions.

It is therefore an object of the present invention a method for treating diarrhea caused by the interaction of a chemotherapeutic agent with a CFTR protein, which comprises administering sequentially, separately or simultaneously with said chemotherapeutic agent a therapeutically effective amount of a CFTR protein inhibitor to a patient in need of the treatment of such a diarrhea.

It is another object of the present invention a method for treating a cancer sensitive to a potential diarrheagenic chemotherapeutic agent, which comprises administering a therapeutically effective amount of a CFTR protein inhibitor for treating diarrhea occurring when said chemotherapeutic agent is administered to a patient.

According to the present invention the definition “chemotherapeutic agent” includes diarrheagenic chemotherapeutic agents, i.e. chemotherapeutic agents which may potentially induce diarrhea when administered to a patient in need thereof such as, for example, fluoropyrimidines, e.g. 5-fluorouracil (5-FU), platinum derivatives, e.g. cisplatin and oxaliplatin, thymidylate synthase inhibitors e.g. raltitrexed and camptothecin derivatives, e.g. irinotecan, SN-38, rubitecan and topotecan.

Preferred examples of diarrheagenic chemotherapeutic agents according to the invention are camptothecin derivatives, particularly irinotecan, SN-38 and topotecan.

According to the present invention, the term “CFTR inhibitor” includes small molecules such as glyburide (glibenclamide), thiazolidinones such as for example 3-[(3-trifluoromethyl)phenyl[-5-[(4-carboxyphenyl)methylene]-2-thioxo-4-thiazolidinone, flavinoids and/or monoclonal or polyclonal antibodies directed toward some part of CFTR (ABCC7).

In a particular aspect, the present invention provides a method for treating diarrhea which results from the interaction of a camptothecin derivative, particularly selected from the group consisting of irinotecan, SN-38 and topotecan, with a CFTR protein, which comprises administering sequentially, separately or simultaneously with said chemotherapeutic agent a therapeutically effective amount of a CFTR protein inhibitor to a patient in need of the treatment of such a diarrhea.

In a more particular aspect, the present invention provides a method for treating diarrhea which results from the interaction of irinotecan or SN-38, with a CFTR protein, which comprises administering sequentially, separately or simultaneously with said chemotherapeutic agent a therapeutically effective amount of a CFTR protein inhibitor to a patient in need of the treatment of such a diarrhea.

According to the present invention the term “treating” includes, unless otherwise specified, preventing, controlling, ameliorating, alleviating, decreasing, reducing and inhibiting.

As an example, the efficacy of a CFTR inhibitor for the treatment of diarrhea induced by the administration of a chemotherapeutic agent, such as for example irinotecan or SN-38, may be evaluated in CFTR knockout mice.

It is believed that the subject CFTR inhibitor would be found to be effective in the treatment of diarrhea induced by the administration of the selected diarrheagenic chemotherapeutic agent.

Standardized methods for assessing diarrhea in cancer patients are needed to better manage CID. The initial assessment and management of CID should include an initial comprehensive and accurate clinical assessment, subsequent targeted reassessment, and the administration of appropriate pharmacological therapies at the optimal time and dosages.

Since diarrhea can unbalance chloride levels in a patients undergoing diarrheagenic chemotherapy, monitoring alteration of chloride levels, for example by detecting chloride levels pre- and post-administration of the chemotherapeutic agent may help the physician to evaluate/predict the patient sensitivity to said diarrheagenic chemotherapeutic agents and hence optimizing time and dosages of the same.

Chloride levels can be monitored, for example, through the detection of chloride in a sweat sample of the patient.

It is therefore another aspect of the present invention a method for assessing diarrhea in a cancer patient undergoing a diarrheagenic chemotherapy, which comprises monitoring chloride levels in said patient, in particular by detecting chloride levels pre- and post-administration of a chemotherapeutic agent.

It is a still another aspect of the present invention a method for optimizing time and dosages of a diarrheagenic chemotherapeutic agent in a patient in need thereof, which comprises evaluating the sensitivity of said patients towards said agent through the detection of chloride levels in a biological sample of said patient and selecting a time and dosages of said agent based on the above chloride levels.

Detection of chloride levels in a biological sample of a patient may be obtained, for example, by detecting chloride in a sweat sample of the patient, following a procedure known in the art.

It is a further another aspect of the present invention a method for optimizing the administration of a diarrheagenic chemotherapeutic agent, which comprises:

(a) obtaining a sweat sample before and subsequently the administration of said chemotherapeutic agent to a patient in need thereof;

(b) detecting the amount of chloride in said samples; and

(c) selecting a therapeutically effective amount of said drug based on the above chloride levels.

It is a still further aspect of the present invention a method for treating a cancer sensitive to a diarrheagenic chemotherapeutic agent, which comprises:

(d) obtaining a sweat sample before and subsequently the administration of said chemotherapeutic agent to a patient in need thereof;

(e) detecting the amount of chloride in said samples; and

(f) selecting a therapeutically effective amount of said drug based on the above chloride levels.

1. A method for treating diarrhea caused by the interaction of a diarrheagenic chemotherapeutic agent with a CFTR protein, which comprises administering sequentially, separately or simultaneously with said chemotherapeutic agent a therapeutically effective amount of a CFTR protein inhibitor to a patient in need of the treatment of such a diarrhea.

2. The method of claim 1, wherein the chemotherapeutic agent is selected from the group consisting of fluoropyrimidines, platinum derivatives, thymidylate synthase inhibitors and camptothecin derivatives.

3. The method of claim 2, wherein the camptothecin derivative is selected from the group consisting of irinotecan, SN-38, rubitecan and topotecan.

4. The method of claim 3 wherein the camptothecin derivative is irinotecan.

5. A method for treating a cancer sensitive to a potential diarrheagenic chemotherapeutic agent, which comprises administering a therapeutically effective amount of a CFTR protein inhibitor for treating diarrhea occurring when said chemotherapeutic agent is administered to a patient.

6. The method of claim 5, wherein the chemotherapeutic agent is selected from the group consisting of fluoropyrimidines, platinum derivatives, thymidylate synthase inhibitors and camptothecin derivatives.

7. The method of claim 6, wherein the camptothecin derivative is selected from the group consisting of irinotecan, SN-38, rubitecan and topotecan.

8. The method of claim 7 wherein the camptothecin derivative is irinotecan.

9. A method for assessing diarrhea in a cancer patient undergoing a diarrheagenic chemotherapy, which comprises monitoring chloride levels in said patient, in particular by detecting chloride levels pre- and post-administration of a chemotherapeutic agent.

10. A method for optimizing time and dosages of a diarrheagenic chemotherapeutic agent in a patient in need thereof, which comprises evaluating the sensitivity of said patients towards said agent through the detection of chloride levels in a biological sample of said patient and selecting a time and dosages of said agent based on the above chloride levels.

11. A method for optimizing the administration of a diarrheagenic chemotherapeutic agent, which comprises:
(a) obtaining a sweat sample before and subsequently the administration of said chemotherapeutic agent to a patient in need thereof;
(b) detecting the amount of chloride in said samples; and
(c) selecting a therapeutically effective amount of said drug based on the above chloride levels.

12. A method for treating a cancer sensitive to a diarrheagenic chemotherapeutic agent, which comprises:
(d) obtaining a sweat sample before and subsequently the administration of said chemotherapeutic agent to a patient in need thereof;
(e) detecting the amount of chloride in said samples; and
(f) selecting a therapeutically effective amount of said drug based on the above chloride levels.
